(19) United States
(12) Patent Application Publication
(10) Pub. No.: US 2021/0260225 A1
(43) Pub. Date: Aug. 26, 2021

(54) PROTECTION OF BIOLOGICALLY ACTIVE MOLECULES DURING RADIATION STERILIZATION

(71) Applicant: Qiagen Sciences LLC, Germantown, MD (US)

(72) Inventors: Paul Q. Hu, Frederick, MD (US); Nadia Patrice Allen, Montgomery Village, MD (US); Jenny Louise Howard, Germantown, MD (US); Jeff Boyle, Germantown, MD (US)

(21) Appl. No.: 17/056,393

(22) PCT Filed: May 17, 2019
(86) PCT No.: PCT/US2019/032885

§ 371 Date: Nov. 17, 2020

Related U.S. Application Data
(60) Provisional application No. 62/673,671, filed on May 18, 2018.

(51) Int. Cl. A61L 2/00; G01N 33/543

(52) U.S. Cl. CPC A61L 2/0035; G01N 33/54393; A61L 2/007

(57) ABSTRACT

Compositions and methods are disclosed that relate to protecting biological activity of a biologically active molecule, including a biologically active protein or biological response modifier such as an immune response modifier, against radiation damage during radiation sterilization. Inclusion of at least one radio-protectant compound, for example, cysteine, reduced glutathione, melatonin, and/or histidine, in an exemplary mitogenic lectin formulation during spray-drying onto surfaces of immunoassay tubes, surprisingly protected the lectin against loss of biological (mitogenic) activity that would otherwise result from electron beam radiation sterilization. The radioprotectant compound also protected other biologically active molecules and stabilized their biological activities, permitting them to retain biological activity after extended storage following the radiation treatment.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Provisional Patent Application No. 62/673,671, filed May 18, 2018, are incorporated herein by reference, in their entirety. Aspects of the embodiments can be modified, if necessary to employ concepts of the various patents, applications and publications to provide yet further embodiments.

### BACKGROUND

### Technical Field

The present embodiments relate generally to in vitro biological assays. More specifically, the present disclosure relates to compositions and methods that preserve, protect, and/or restore and/or stabilize the biological activity of biologically active molecules, including but not limited to biologically active proteins and/or biological response modifiers such as immune response modifiers, the activities of which would otherwise be compromised and/or diminished during and/or after radiation sterilization.

### Description of the Related Art

In vitro assays of immunologic activity of cells such as immune system cells present in a biological sample obtained from a patient (e.g., lymphocytes, monocytes, macrophages, dendritic cells, or other cells of the immune system) are well known in the art for diagnostic and prognostic purposes. For instance, whole blood samples or white blood cells separated from such samples may be tested in vitro to assess immune response capability by a number of measurements, such as cell proliferation, soluble mediator release and/or activation in response to stimulation by mitogens, by specific antigen(s), or by pathogen-associated molecular patterns (PAMPs), receptors and other agonists.

Mitogens include proteins that are known to stimulate cellular mitosis and are used as immunological reagents to induce lymphocyte proliferation in a non-antigen-specific fashion. Lymphocyte mitogens can be polyclonal activators that stimulate lymphocytes to proliferate and/or to release/secrete soluble mediators, and may do so by engaging non-antigen-stimulation driven lymphocyte molecular mechanisms whilst bypassing antigen-specific receptors (immunoglobulins (Ig) or T-cell receptors (TCR) to elicit a robust polyclonal response that can be readily detected. Exemplary T-cell mitogens include the biologically active proteins phytohemagglutinin (PHA) and concanavalin A (ConA), which are lectins (e.g., plant-derived carbohydrate-binding proteins). Another lectin, pokeweed mitogen (PWM), is capable of stimulating both T-cells and B-cells. Mitogenic lectins from a variety of sources have been described (e.g., Shanmugham et al., 2006 Riv Biol. 99:227; Naeem et al., 2007 Curr. Protein Pept. Sci 8:261; Singh et al., 2014 Crit. Rev. Microbiol. 40:329). Certain antibodies that specifically bind to lymphocyte cell surface molecules capable of activation signal transduction may also function as mitogens.

Mitogens are therefore particularly useful to assess the overall immunoresponsiveness in a lymphocyte-containing sample by eliciting/stimulating robust polyclonal responses that can be readily measured in samples in which the immunodetections of monoclonal or oligoclonal responses may not be sufficiently sensitive due to low signal strength or low frequencies of antigen-specific responding lymphocytes.

The QuantiFERON® TB Gold Mitogen Control assay (e.g., Mazurek et al., 2005 MMWR Recomm. Rep 54:49-55; Mazurek et al., 2010 MMWR Recomm. Rep. 59:1-25; Simpson et al., 2012 Heart Lung 41:553; Cho et al., 2012 Tuberc. Respir. Dis. (Seoul) 72:416; Woo et al., 2014 Clin. Chim. Acta 430:79), for example, employs the biologically active protein PHA (a lectin) as a polyclonal mitogen to induce robust in vitro T-cell responses, from which immunoresponsive status of T-cells present in a sample can be assessed. In this test, PHA is conveniently provided in dried form as a coating on the inner surfaces of blood collection tubes. The mitogen-coated tubes are prepared by spray-drying a PHA-containing solution on the inner surfaces, followed by radiation sterilization (e.g., electron beam radiation, gamma-irradiation, etc.) to kill or inactivate any potential microbial contaminants that may be present. The use of chemicals instead of radiation to achieve a sterile environment may not be ideal, as chemicals can interfere with the biological activity of the cells during the stimulation and/or interfere with other assay detection systems. Electron beam radiation is a standard technique known in the art for such radiation sterilization (e.g., Smith et al., 2016 Health Phys. 111(2 Suppl 2):S141; Silindir et al., 2012 PDA J Pharm Sci Technol. 66:184; Mehta et al., 1993 Med Device Technol. 4:24; Yaman, 2001 Curr. Opin. Drug Devel. 4:760) of biologically active proteins.

It is desirable to have a sterile environment in the blood collection/culture tubes so that the immune responsiveness of lymphocytes in the biological sample (e.g., whole blood or isolated peripheral blood white cells) following exposure to known biologically active assay components (e.g., a mitogenic protein such as PHA) is not altered or obscured by a cellular response to microbial contaminants present in the assay/culture tube. Furthermore, as blood collection tubes can come in direct contact with the blood of a patient, there is a risk that an infectious agent could be passed on to the patient if the tube contents are not sterile.

Electron beam radiation sterilization has, however, been reported to significantly alter the structural, biological and/or immunological properties of proteins, thereby raising concerns about potentially detrimental effects of such radiation on biological and biomedical products (e.g., Katial et al., 2002 J Allerg Clin Immunol 110:215; Terryn et al., 2007 Int J Pharm 343:4; Antebi et al., 2016 Rev Bras Ortop 51:224). For example, electron beam sterilization may dramatically diminish the potency and affect the lot-to-lot consistency of protein preparations within the sterile immunoassay/culture tubes, such as PHA formulations in QuantiFERON® TB Gold Mitogen blood collection tubes. As a consequence, production efficiency will be decreased due to diminution of the bioactivity of an active ingredient (e.g., a mitogen) as a result of the terminal radiation sterilization. Increased input amounts of raw materials (e.g., mitogenic protein) used in the production of collection tubes are thus required to compensate for the loss of bioactivity, but the need for such increased amounts will result in increased production costs and undesirable variability among different production batches.

Various publications teach methods to protect biological tissues, cells and/or biomolecules from radiation damage by modifying solvent content, temperature, pH, oxygen content or other parameters during radiation sterilization and/or by using various stabilizers during sterilization procedures. From these disclosures, it is apparent that the practicability of, compatibility with, and radioprotection of any particular biomolecule or class of biomolecules by a given stabilizer or combination of stabilizers, or achievement of radioprotection by modifying other conditions, cannot be predicted but must instead be determined empirically for the biomolecules that are desirably to be protected.

For example, EP2236520 describes stabilization of biomolecules, including stabilization to protect against harmful effects of electromagnetic radiation, under conditions selected to avoid freezing the biomolecules. Stabilizers include the use of a required minimum of at least two different amino acids and as many as 18 different amino acids, with combinations of two to five different amino acids being preferred. U.S. Pat. No. 5,730,933 describes protection of biomolecules from radiation damage by contacting them with an extraneous protein (e.g., bovine serum albumin or denatured collagen) and a free-radical scavenger/antioxidant and freezing prior to irradiation, optionally with a lyophilization step. U.S. Pat. No. 6,946,098 describes addition of human serum albumin (HSA) to biologicals as a stabilizer, followed by radiation sterilization to destroy prions, viruses or other pathogens. An extensive listing of alternative stabilizer agents is disclosed including fatty acids, antioxidants, free-radical scavengers, heparin, and thiol compounds, but only HSA is described in the worked Examples.

US20030012687 and US20030031584 describe radioprotection of tissues or biomolecules such as immunoglobulins using stabilizers drawn from a wide variety of classes of compounds, including fatty acids, free-radical scavengers, antioxidants, sugars, selected amino acids and dipeptides, Trolox (CAS 53188-07-1), and others. US20030143106 and US20040086420 describe radioprotection of tissues and of various blood, serum and plasma proteins using a variety of antioxidants, fatty acids, amino acids, vitamins, and/or free-radical scavengers. US20050069453 describes protection of urokinase during radiation sterilization by modifying the sample properties (e.g., solvent composition, pH, temperature, etc.) or by adding any of an extensive list of stabilizing agents, only a small number of which are demonstrated to confer radioprotection in the worked Examples.

Clearly there is a need to protect the biological activity of biologically active proteins and other biologically active molecules against radiation damage during radiation sterilization, to improve immunoassay product quality and consistency, and to reduce the amount of raw material required for immunoassay/culture tube/system production. The presently disclosed invention embodiments address these needs and offer other related advantages.

### BRIEF SUMMARY

According to certain aspects of the presently disclosed invention, there is provided a method of protecting biological activity of a biologically active protein or other biologically active molecule against radiation damage during radiation sterilization, comprising: (a) contacting the biologically active protein or other biologically active molecule in an aqueous solution with at least one soluble radioprotectant compound to obtain a radioprotected mixture prior to radiation sterilization; and (b) radiation sterilizing the radioprotected mixture, wherein biological activity of the biologically active protein or other biologically active molecule in the radioprotected mixture after radiation sterilization is greater than biological activity of a control sample of the biologically active protein or other biologically active molecule that is radiation sterilized without the radioprotectant compound present, and thereby protecting biological activity of the biologically active protein or other biologically active molecule against radiation damage during radiation sterilization. In a further embodiment, the radioprotected mixture is dried prior to the step of radiation sterilizing.

In another embodiment there is provided a method of protecting a plurality of molecules of a biologically active protein or other biologically active molecule against a loss of biological activity from said plurality of molecules during a period of time in storage, comprising (a) contacting the biologically active protein or other biologically active molecule in an aqueous solution with at least one soluble radioprotectant compound to obtain a radioprotected mixture prior to radiation sterilization; (b) drying the radioprotected mixture to obtain a dried radioprotected mixture; (c) radiation sterilizing the dried radioprotected mixture; and (d) storing the dried radioprotected mixture for a period of time to obtain a stored dried radioprotected mixture, wherein biological activity of the biologically active protein or other biologically active molecule in the stored dried radioprotected mixture after radiation sterilization and storage for said period of time is greater than biological activity of a control sample of the biologically active protein or other biologically active molecule that is dried, radiation sterilized without the radioprotectant compound present, and then stored for the period of time, and thereby protecting a plurality of molecules of the biologically active protein or other biologically active molecule against loss of biological activity during the period of time in storage.

In certain other embodiments there is provided a method of protecting biological activity of a biologically active protein or other biologically active molecule such as a biologically active imidazoquinoline having TLR agonist activity against radiation damage during radiation sterilization, comprising (a) contacting the biologically active protein or other biologically active molecule, for example, the biologically active imidazoquinoline having TLR agonist activity, in an aqueous solution with at least one soluble radioprotectant compound to obtain a radioprotected mixture prior to radiation sterilization; (b) drying the radioprotected mixture to obtain a dried radioprotected mixture; and (c) radiation sterilizing the dried radioprotected mixture to obtain a dried radiation sterilized radioprotected mixture, wherein, following rehydration of the dried radiation sterilized radioprotected mixture to obtain a rehydrated radiation sterilized radioprotected mixture, biological activity of the biologically active protein or other biologically active molecule (e.g., the biologically active imidazoquinoline) in the radioprotected mixture after radiation sterilization is greater than biological activity of a control sample of the biologically active protein or other biologically active molecule (e.g., biologically active imidazoquinoline) that is radiation sterilized without the radioprotectant compound present, and thereby protecting biological activity of the biologically active protein or other biologically active molecule (e.g., biologically active imidazoquinoline) against radiation damage during radiation sterilization. In certain further embodiments the biologically active imidazoquinoline having TLR agonist activity comprises one or more of imiquimod, gardiquimod, and resiquimod (R848).

In certain further embodiments of the above described methods, the biologically active protein is a mitogen, which in certain still further embodiments is selected from phytohemagglutinin (PHA), concanavalin A (ConA), and pokeweed mitogen (PWM). In certain other further embodiments of the above described methods, the biologically active protein comprises one or more of (i) a mitogen, (ii) an antibody, (iii) an enzyme, (iv) a cytokine, (v) a growth factor, and (vi) a hormone. In certain embodiments the radioprotectant compound comprises at least one antioxidant compound, which in certain further embodiments is selected from cysteine, glutathione and melatonin. In certain embodiments the radioprotectant compound comprises histidine. In certain embodiments of the above described methods, the radioprotectant compound is present in the radioprotected mixture at a concentration of at least 0.1, 0.2, 0.3, 0.5, 0.7, 1.0, 2, 3, 4, 5, 6, 7, 8, 9, 10, 11, 12, 13, 14, 15, 16, 17, 18, 19, 20, or 50 millimolar. In certain embodiments the biological activity comprises mitogenic activity, which in certain further embodiments comprises lymphocyte proliferation inducing activity. In certain still further embodiments the lymphocyte proliferation activity comprises T-cell proliferation inducing activity.

These and other aspects and embodiments of the herein described invention will be evident upon reference to the following detailed description and attached drawings. All of the U.S. patents, U.S. patent application publications, U.S. patent applications, foreign (non-U.S.) patents, foreign patent applications and non-patent publications referred to in this specification and/or listed in the Application Data Sheet are incorporated herein by reference in their entirety as if each was incorporated individually. Aspects and embodiments of the invention can be modified, if necessary, to employ concepts of the various patents, applications and publications to provide yet further embodiments.

### BRIEF DESCRIPTION OF THE SEVERAL VIEWS OF THE DRAWINGS

FIG. 1 shows concentration-dependent protection of biological activity in a PHA-P formulation treated with electron beam radiation sterilization at 25 kGy in solution. Cysteine (closed circles) and melatonin (open circles) were dissolved in the same PHA-P formulation to various final concentrations. The mitogenic activities were determined by measuring IFN-γ secretion in whole blood samples from six blood donors using QuantiFERON® ELISA according to the manufacturer's instructions (QIAGEN, Inc., Germantown, Md.). Group mean percentages of mitogenic activity over controls (PHA-P alone without any additives) are presented. Results demonstrated dose dependent protection of the mitogenic activities of cysteine and melatonin in solution.

FIG. 2 shows protection of mitogen potency against the loss of biological activity of spray dried PHA-P in a QuantiTFERON® Mitogen Control blood collection tube during electron beam radiation sterilization. QuantiFERON® Mitogen Control blood collection tubes were manufactured with a mitogen (PHA) formulation supplemented with cysteine (5 mM final concentration in liquid mitogen formulation). Mitogen responses were determined in mitogen tubes formulated with and without cysteine, treated and untreated with electron beam radiation sterilization, in a group of eight donors. Data were normalized to the % response of control which was the non-sterilized control mitogen tube (without cysteine). Each dot in the figure represents the data point from one donor. Results demonstrated that cysteine did not affect the mitogen response but protected the mitogenic activity of spray dried PHA-P in E-Beam radiation sterilization.

FIG. 3 shows the percentages of differences in mitogen potency in QuantiFERON® Mitogen Control blood collection tubes produced with and without 5 mM cysteine in PHA followed by electron beam radiation sterilization and stored for the indicated number of months of post-production. QuantiFERON® Mitogen Control blood collection tubes were produced with and without cysteine (5.0 mM final concentration in liquid mitogen formulation) and sterilized with E-Beam radiation. Mitogen tubes' biological (mitogenic) activities were tested over time and are presented as % potency differences of the activity of mitogen tubes formulated with cysteine compared to mitogen tubes formulated without cysteine. Each dot in the figure represents the % activity of group mean at each time point. Results demonstrated that mitogen tubes formulated with cysteine had higher % activity than the tubes formulated without cysteine over time, indicating that mitogen tubes with cysteine had less loss of activity over time than the tubes without cysteine.

FIG. 4 shows protection of the T-cell stimulatory activity of anti-CD3 antibody against E-beam radiation by the radioprotectant compounds cysteine (Cys) or glutathione (G-SH), as assessed by IFN-γ secretion in whole blood samples from six donors.

FIG. 5 shows the effects of titrating the radioprotectant compounds cysteine (Cys) or glutathione (G-SH) on protection of the T-cell stimulatory activity of anti-CD3 antibody against E-beam radiation, as assessed by IFN-γ secretion in whole blood samples from six donors.

FIG. 6 shows protection of the NK cell stimulatory activity of the TLR agonist imidazoquinoline immune response modifier R848 (resiquimod) against E-beam radiation by the radioprotectant compounds cysteine (Cys) or glutathione (G-SH), as assessed by IFN-γ secretion in whole blood samples from six donors.

FIG. 7 shows the effects of titrating the radioprotectant compounds cysteine (Cys) or glutathione (G-SH) on protection of the NK cell stimulatory activity of the TLR agonist imidazoquinoline immune response modifier R848 (resiquimod) against E-beam radiation, as assessed by IFN-γ secretion in whole blood samples from six donors.

FIG. 8 shows protection of the combined T-cell stimulatory activity of anti-CD3 antibody and NK cell stimulatory activity of the TLR agonist imidazoquinoline immune response modifier R848 (resiquimod) against E-beam radiation by the radioprotectant compounds cysteine (Cys) or glutathione (G-SH), as assessed by IFN-γ secretion in whole blood samples from six donors.

FIG. 9 shows the effects of titrating the radioprotectant compounds cysteine (Cys) or glutathione (G-SH) on protection of the combined T-cell stimulatory activity of anti-CD3 antibody and NK cell stimulatory activity of the TLR agonist imidazoquinoline immune response modifier R848 (resiquimod) against E-beam radiation, as assessed by IFN-γ secretion in whole blood samples from six donors.

FIG. 10 shows protection of the mitogenic potency of pokeweed mitogen (PWM) against E-beam radiation by the radioprotectant compounds cysteine (Cys) or glutathione (G-SH), as assessed by IFN-γ secretion in whole blood samples from six donors.

FIG. 11 shows the effects of titrating the radioprotectant compounds cysteine (Cys) or glutathione (G-SH) on protection of the mitogenic potency of pokeweed mitogen (PWN) against E-beam radiation, as assessed by IFN-γ secretion in whole blood samples from six donors.

FIG. 12 shows protection of the T-cell stimulatory activity of the T-cell mitogen conconavalin A (ConA) against E-beam radiation by the radioprotectant compounds cysteine (Cys) or glutathione (G-SH), as assessed by IFN-γ secretion in whole blood samples from six donors.

FIG. 13 shows the effects of titrating the radioprotectant compounds cysteine (Cys) or glutathione (G-SH) on protection of the T-cell stimulatory activity of the T-cell mitogen conconavalin A (ConA) against E-beam radiation, as assessed by IFN-γ secretion in whole blood samples from six donors.

### DETAILED DESCRIPTION

Certain presently disclosed embodiments relate to the surprising discovery that biological activity of a biologically active protein or other biologically active molecule such as biological response modifier or an immune response modifier, which would otherwise be compromised by radiation sterilization, may be substantially radioprotected (e.g., increased in a statistically significant manner relative to an appropriate control) if the biologically active protein or other biologically active molecule is contacted with a radioprotectant compound as provided herein, prior to radiation sterilization.

More specifically, and by way of non-limiting example, as described herein the mitogenic activity of PHA toward T lymphocytes was found to be significantly diminished by radiation sterilization of immunoassay tubes in which a PHA solution had been spray-dried. As disclosed herein for the first time, however, if the PHA was contacted with at least one radioprotectant compound as provided herein prior to radiation sterilization, for example, one or more radioprotectant compounds such as cysteine, reduced glutathione, melatonin, and/or histidine, then the PHA biological activity—i.e., mitogenic activity for white blood cells present in human whole blood sample—after radiation sterilization was surprisingly greater than the activity of a control PHA sample that had been radiation sterilized without the radioprotectant compound present. In addition, and as also disclosed herein for the first time, the protective effect of contacting the biologically active protein (PHA) with the radioprotectant compound in solution to obtain a radioprotected mixture unexpectedly persisted following substantial drying of the mixture, such as by spray-dry and/or freeze-drying (e.g., lyophilization). Moreover, the substantially dried radioprotected mixture exhibited surprisingly long-term stability, with substantial protection of biological activity being demonstrated after over eight months of storage.

These and related embodiments will therefore find uses in a large number of contexts in which it may be desired to provide a biologically active protein or other biologically active molecule (including a biological response modifiers such as an immune response modifier) in a sterile environment, such as a spray-dried, dehydrated and/or dried preparation of the biologically active protein (or other biologically active molecule) alone or on a surface of any type of container (e.g., test tube, assay plate, microwell, culture dish, blood specimen container, bottle, beaker, vial, ampoule, syringe, or any other appropriate container) that may be advantageously radiation sterilized in order to obtain the benefits associated with a sterile environment. Certain preferred embodiments as described herein relate to radioprotected protein mitogens for use in any of a variety of in vitro immunological assays, but the contemplated embodiments are not intended to be so limited such that other biologically active proteins (e.g., immunostimulatory antibodies) or other biologically active molecules (e.g., biological response modifiers such as immune response modifiers, for instance, imidazoquinoline Toll-like receptor (TLR) agonists, for example by way of illustration and not limitation, imiquimod, gardiquimod, resiquimod, etc.) are also envisioned in configurations in which the biologically active protein or biologically active molecule may be advantageously radiation sterilized without substantial loss of biological activity.

### Biological Activity

As described herein, the biological activity of a substance means any activity which can affect any physical or biochemical properties of a biological system, pathway, molecule, or interaction relating to an organism, including for example but not limited to, cells, viruses, bacteria, bacteriophage, prions, insects, fungi, plants, animals, and humans. Examples of substances with biological activity include, but are not limited to, polynucleotides, peptides, proteins and in particular biologically active proteins, including enzymes, antibodies, glycoproteins, lectins, mitogenic proteins including mitogenic lectins, small molecules (e.g., a bioactive small molecule), pharmaceutical compositions (e.g., drugs), vaccines, biological response modifiers including immune response modifiers such as imidazoquinolines having TLR agonist activity (e.g., imiquimod, gardiquimod, resiquimod (R848), etc.), carbohydrates, lipids, steroids, hormones, chemokines, growth factors, cytokines, liposomes, and toxins.

Persons familiar with the relevant art will recognize appropriate assays and methods for determining the biological activity of substances that affect the physical or biochemical properties of a biological system, for example, one or more biological activities that may include, but are not limited to, immunological, immunochemical, cytokine, hormone and bioactive peptide activities and other cell proliferation (e.g., mitogenic) and/or differentiation activities (see for example, Coligan et al. (Eds.) 2007 Current Protocols in Immunology, Wiley and Sons, Inc. Hoboken, N.J.), signal transduction (see for example, Bonifacino et al. (Eds.) 2007 Current Protocols in Cell Biology, Wiley and Sons, Inc. Hoboken, N.J.), immunopotentiation and/or immune response modifier activity such as imidazoquinolines, for example, the TLR agonists imiquimod, gardiquimod, resiquimod (R848), etc. (e.g., Gerster et al., 2005 J. Med. Chem. 48:3481; Shukla et al., 2010 J. Med. Chem. 53:4450; Shi et al., 2012 ACS Med. Chem. Lett. 3(6):501-504; Tomai et al., Ch. 8, Toll-Like Receptor 7 and 8 Agonists for Vaccine Adjuvant Use, pp. 149-161, and Skountzu et al., Ch. 20, Adjuvants for Skin Vaccination, pp. 399-419, in Immunopotentiators in Modern Vaccines, Schijns et al. (eds.), 2017 Academic Press, N.Y.), gene expression (see, e.g., Asubel, F M et al. (Eds.) 2007 Current Protocols in Molecular Biology, Wiley and Sons, Inc. Hoboken, N.J.), receptor-ligand interactions (see for example, Coligan et al. (Eds.) 2007 Current Protocols in Immunology, Wiley and Sons, Inc. Hoboken, N.J.), enzymatic activity (see, e.g., Eisenthal and Hanson (Eds.), Enzyme Assays, Second Edition, Practical Approaches series, No. 257. 2002, Oxford University Press, Oxford, UK; Kaplan and Colowick (Eds.), Preparation and Assay of Enzymes, Methods in Enzymology, (vols. 1, 2 and 6). 1955 and 1961, Academic Press, Ltd., Oxford, UK), and cell toxicity (e.g., cytotoxicity, excitotoxicity) (see for example, Bus J S et al. (Eds) 2007 Current Protocols in Toxicology, Wiley and Sons, Inc. Hoboken, N.J.), apoptosis and necrosis (Green and Reed, 1998 Science 281(5381):1309-12; Green D R, 1998 Nature December 17: 629; Green D R, 1998 Cell 94(6):695-69; Reed, J C (Ed.), 2000 Apoptosis, Methods in Enzymology (vol. 322), Academic Press Ltd., Oxford, UK); or other biological activities.

In preferred embodiments as disclosed herein, there is provided a method for substantially protecting biological activity of a biologically active protein and/or another biologically active molecule (including a biological response modifier such as an immune response modifier, for instance, an imidazoquinoline having TLR agonist activity (e.g., imiquimod, gardiquimod, resiquimod (R848)), against radiation damage during radiation sterilization. In certain further preferred embodiments the biologically active protein is one or a plurality of mitogens, for example, one or more of PHA, ConA, and/or PWM, and/or the biologically active protein comprises one or more of an antibody, a cytokine, an enzyme, a growth factor, and a hormone, and/or the biologically active molecule comprises an immune response modifier that comprises one or a plurality of imidazoquinolines having TLR agonist activity, for example, imiquimod, gardiquimod, and/or resiquimod (R848).

In certain preferred embodiments as disclosed herein, there is provided a method of protecting a plurality of molecules of a biologically active molecule, which in certain preferred embodiments may be a biologically active protein and in certain other preferred embodiments may be a biologically active molecule that comprises one or more biological response modifiers such as immune response modifiers, for instance, imidazoquinoline immune response modifiers having TLR agonist activity, against a loss of biological activity from said plurality of molecules during a period of time in storage, comprising contacting the biologically active protein or other biologically active molecule(s) in an aqueous solution with at least one soluble radioprotectant compound to obtain a radioprotected mixture prior to radiation sterilization; drying the radioprotected mixture to obtain a dried radioprotected mixture; radiation sterilizing the dried radioprotected mixture; and storing the dried radioprotected mixture for a period of time to obtain a stored dried radioprotected mixture, wherein biological activity of the biologically active protein or other biologically active molecule(s) in the stored dried radioprotected mixture after radiation sterilization and storage for said period of time is greater than biological activity of a control sample of the biologically active protein or other biologically active molecule(s) that is dried, radiation sterilized without the radioprotectant compound present, and then stored for the period of time, and thereby protecting a plurality of molecules of the biologically active protein or other biologically active molecule(s) against loss of biological activity during the period of time in storage.

The time period for storage may vary considerably as a function of the particular biologically active molecule(s), the particular biological activity or activities of such molecule(s), the radiation sterilization conditions, the radioprotectant(s), the degree to which the radioprotected mixture is dried, the storage conditions (including, e.g., temperature, relative humidity, ambient atmosphere, etc.), and other factors. Typically the period of time in storage during which the biologically active molecule(s) (e.g., biologically active protein(s)) is protected against a loss of biological activity (e.g., a statistically significant reduction in biological activity relative to an appropriate control) may be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or more months.

Biological activity of a biologically active protein or other biologically active molecule(s) may be substantially protected according to certain herein disclosed embodiments when, following radiation sterilization of a composition that comprises the biologically active protein or other biologically active molecule(s), there is complete recovery of the biological activity, or substantial recovery (e.g., recovery of at least 30, 32, 34, 36, 38, 40, 42, 44, 46, 48, or 50 percent, preferably at least 52, 54, 56, 58, or 60 percent, more preferably at least 62, 64, 66, 68, or 70 percent, more preferably at least 72, 74, 76, or 80 percent, and typically in more preferred embodiments at least 81, 82, 83, 84, or 85 percent, more preferably at least 86, 87, 88, 89, 90, 91, 92, 93 or 94 percent, more preferably at least 95 percent, still more preferably greater than 96, 97, 98 or 99 percent) of the biological activity.

In certain embodiments that are described herein, a biologically active protein or other biologically active molecule (including a biologically active biological response modifier such as an immune response modifier, for instance, an imidazoquinoline immune response modifier having TLR agonist activity) in an aqueous solution is contacted with at least one soluble radioprotectant compound to obtain a radioprotected mixture prior to radiation sterilization.

Radiation sterilization of the radioprotected mixture may be achieved according to any of a number of known procedures, for instance, electron beam radiation as described by, e.g., Smith et al., 2016 Health Phys. 111(2 Suppl 2):S141; Silindir et al., 2012 PDA J Pharm Sci Technol. 66:184; Mehta et al., 1993 Med Device Technol. 4:24; Yaman, 2001 Curr. Opin. Drug Devel. 4:760; Katial et al., 2002 J Allerg Clin Immunol 110:215; Terryn et al., 2007 Int J Pharm 343:4; and Antebi et al., 2016 Rev Bras Ortop 51:224.

In certain preferred embodiments, the radioprotected mixture is dried or substantially dried prior to radiation sterilization, which typically may be complete drying (e.g., with statistical significance, all or substantially all detectable solvent has been removed). In certain embodiments which may vary according to the nature of the sample to be stored and its intended uses, greater than 75%, 80%, 82%, 84%, 86%, 88%, 90%, 91%, 92%, 93%, 94%, 95%, 96%, 97%, 98% or 99% of detectable solvent has been removed for purposes of obtaining a dried, dry, substantially dried, or substantially dry radioprotected mixture.

After the step of contacting the biologically active protein or other biologically active molecule as provided herein with the radioprotectant compound to obtain the radioprotected mixture, the radioprotected mixture may be dried according to any of a variety of drying methodologies. A preferred drying method is lyophilization (e.g., freeze-drying, such as drying a frozen aqueous solution under a partial or complete vacuum to promote removal of water by sublimation from the frozen solid state to the vapor phase without formation of liquid water). Other drying techniques may also be employed, for example, drying by evaporation of solvent (e.g., water) at ambient temperature and pressure, or in a laminar flow hood or desiccating chamber, or under reduced atmospheric pressure including under vacuum (e.g. with vacuum pump such as a SpeedVac®). Other methods of drying are also contemplated and include for example without limitation, radiant heat drying, drying under a light source, desiccating, drying under nitrogen or other gas (e.g., preferably under a stream of a flowing inert gas), use of drying solvents or other chemicals, for example, volatile organic solvents such as lower alcohols, lower alkanes and haloalkanes (e.g., pentanes, hexanes, methylene chloride, chloroform, carbon tetrachloride), ethers (e.g., tetrahydrofuran), ethyl acetate, acetonitrile, trifluoroacetic acid, pyridine, acetone or other solvents (preferably in anhydrous form), air pressure, and other methods to facilitate and accelerate evaporation.

Drying of the sample can be determined by simple visual inspection or touch (i.e. tapping with a pipette tip) to ensure all moisture has been evaporated or removed. In some embodiments, a moisture indicator may be preferably included to ascertain a degree of drying that has been achieved. For example, cobalt chloride may optionally be included as a detectable (by visible color-change or colorimetry) indicator of moisture content in a sample. A moisture indicator such as an electronic device that measures the dielectric content of material to determine moisture content (e.g., Aqua-Spear™, Mastrad Limited, Douglas, UK) is also contemplated for use in certain of these and related embodiments. A drying agent such as calcium sulfate (i.e., Drierite®, W.A. Hammond Drierite Co., Xenia, Ohio) or phosphorus pentoxide with a moisture indicator is also contemplated for use in certain embodiments of the present disclosure.

### Radioprotectant Compounds

As also described elsewhere herein, the present disclosure relates to the unexpected discovery that the biological activity of a biologically active molecule as provided herein, such as a biologically active protein, which activity would otherwise be compromised by radiation sterilization, may be substantially radioprotected (e.g., increased in a statistically significant manner relative to the biological activity of an unprotected appropriate control, such as that of the same biologically active molecule (e.g., biologically active protein) that has undergone radiation sterilization in the absence of a radioprotectant) if the biologically active molecule is contacted with a radioprotectant compound as provided herein, prior to radiation sterilization, to form a radioprotected mixture that undergoes radiation sterilization.

Despite previous observations (e.g., as summarized above) of certain radioprotective effects conferred by certain stabilizing agents to preserve or partially preserve at least some structural and/or functional attributes of biological tissues, cells, or biological molecules, the skilled artisan would not reasonably have expected to arrive at the presently disclosed combination of features.

Thus, there is disclosed for the first time herein a method of substantially protecting biological activity of a biologically active protein or other biologically active molecule against radiation damage during radiation sterilization, comprising contacting the biologically active protein or other biologicall active molecule in an aqueous solution with at least one soluble radioprotectant compound to obtain a radioprotected mixture prior to radiation sterilization; and radiation sterilizing the radioprotected mixture, wherein biological activity of the biologically active protein or other biologically active molecule in the radioprotected mixture after radiation sterilization is greater (e.g., increased in a statistically significant manner relative to an appropriate control) than biological activity of a control sample of the biologically active protein or other biologically active molecule that is radiation sterilized without the radioprotectant compound present, and thereby substantially protecting biological activity of the biologically active protein or other biologically active molecule against radiation damage during radiation sterilization. In certain further embodiments the radioprotected mixture is substantially dried prior to the step of radiation sterilizing.

Also disclosed for the first time herein is a method of substantially protecting biological activity of a biologically active protein or other biologically active molecule against radiation damage during radiation sterilization, comprising contacting the biologically active protein or other biologically active molecule in an aqueous solution with at least one soluble radioprotectant compound to obtain a radioprotected mixture prior to radiation sterilization; substantially drying the radioprotected mixture to obtain a substantially dry radioprotected mixture; and radiation sterilizing the substantially dry radioprotected mixture to obtain a substantially dry radiation sterilized radioprotected mixture, wherein, following rehydration of the substantially dry radiation sterilized radioprotected mixture to obtain a rehydrated radiation sterilized radioprotected mixture, biological activity of the biologically active protein or other biologically active molecule in the radioprotected mixture after radiation sterilization is greater (e.g., increased in a statistically significant manner relative to an appropriate control) than biological activity of a control sample of the biologically active protein or other biologically active molecule that is radiation sterilized without the radioprotectant compound present, and thereby substantially protecting biological activity of the biologically active protein or other biologically active molecule against radiation damage during radiation sterilization.

More particularly, according to the present disclosure it is demonstrated for the first time that the biological activity of a biologically active protein, such as PHA, ConA or PWM, or anti-CD3 antibody, which acts as a mitogen for human and other mammalian peripheral blood lymphocytes, is sensitive to electron beam radiation sterilization and is decreased (e.g., reduced in a statistically significant manner relative to an appropriate control) relative to the activity of the same mitogen that has not undergone radiation sterilization. Furthermore, and as also disclosed herein for the first time, the biological activity of such a mitogen can be protected (e.g., increased in a statistically significant manner relative to an appropriate control) from the compromising effects of electron beam radiation by being contacted with at least one radioprotectant compound as provided herein to form a radioprotected mixture that is then subjected to radiation sterilization.

The present disclosure also teaches for the first time that in the methods described herein, the radioprotectant compound that confers bioactivity protection on the present mitogens may comprise (or consist of) one or more of cysteine, melatonin, glutathione and histidine.

Still further, the present disclosure teaches for the first time that the presently provided radioprotectant compound (e.g., as may comprise or consist of one or more of cysteine, melatonin, glutathione and histidine) can be combined with the present biologically active protein mitogen (e.g., PHA, ConA and/or PWM, or anti-CD3 antibody) or immune response modifier (e.g., imidazoquinoline having TLR agonist activity such as imiquimod, gardiquimod, resiquimod (R848), etc.) to form a radioprotected mixture that can be substantially dried (e.g., lyophilized) as provided herein to undergo radiation sterilization as a substantially dry radioprotected mixture, wherein even in such dried form the presence of the radioprotectant compound preserves biological activity of the mitogen (e.g., which activity is increased in a statistically significant manner when compared to an appropriate control) relative to the mitogenic activity of a control sample from which the radioprotectant compound is omitted.

The present disclosure therefore teaches radioprotection of mitogens and other biologically active molecules by a method that the art previously failed to appreciate, using radioprotectant compounds that would not previously have been expected to have such capabilities, including protective ability when present along with the mitogen in the form of a substantially dry radioprotected mixture as described herein. For instance, agents previously recognized as having radioprotective properties in solution for proteins other than the present mitogens are described herein as surprisingly exhibiting radioprotective effects toward different proteins (e.g., the instant mitogens) when present along with the mitogen in a different physical state (e.g., as a lyophilized substantially dry radioprotected mixture instead of in solution) as described herein. These properties would not have been predicted prior to the present disclosure.

Accordingly and in certain preferred embodiments, one or more biologically active protein(s) such as a herein described mitogen, for instance, anti-CD3 antibody, PHA, ConA and/or PWM, and/or one or more biologically active immune response modifier(s) such as a herein described imidazoquinoline TLR agonist, for instance, imiquimod, resiquimod (R848) and/or gardiquimod, may be contacted with at least one soluble radioprotectant compound, for example, cysteine, glutathione, melatonin, and/or histidine, to permit the drying of the biologically active protein(s) and/or immune response modifier(s) and the radioprotectant compound(s) to proceed at the same time, thereby to obtain a substantially dry radioprotected mixture, which may then be radiation sterilized to obtain a substantially dry radiation sterilized radioprotected mixture.

In certain preferred embodiments, one or more biologically active protein(s) may include antibodies to cell surface receptors, for instance, antibodies or antigen-binding fragments thereof that specifically bind to CD3, OX40, CD40L, CD152 and/or CD28, which antibodies or antigen-binding fragments thereof may be contacted with at least one soluble radioprotectant compound, for example, cysteine, glutathione, melatonin, and/or histidine, to permit the drying of the biologically active protein(s) and the radioprotectant compound(s) to proceed at the same time, thereby to obtain a substantially dry radioprotected mixture, which may then be radiation sterilized to obtain a substantially dry radiation sterilized radioprotected mixture.

In certain other preferred embodiments, one or more biologically active protein(s) may include antigens, for instance, peptides or proteins that can be recognized in specific binding interactions by selective elements of the adaptive immune system (e.g., antibodies or antigen-binding fragments thereof, T-cell receptors or antigen-binding fragments thereof, etc.), which antigens may be contacted with at least one soluble radioprotectant compound, for example, cysteine, glutathione, melatonin, and/or histidine, to permit the drying of the biologically active protein(s) and the radioprotectant compound(s) to proceed at the same time, thereby to obtain a substantially dry radioprotected mixture, which may then be radiation sterilized to obtain a substantially dry radiation sterilized radioprotected mixture.

In certain other preferred embodiments, one or more biologically active protein(s) may include cytokines, for instance, TNF-α, IFN-γ, IL-1, IL-2, etc., which may be contacted with at least one soluble radioprotectant compound, for example, cysteine, glutathione, melatonin, and/or histidine, to permit the drying of the biologically active protein(s) and the radioprotectant compound(s) to proceed at the same time, thereby to obtain a substantially dry radioprotected mixture, which may then be radiation sterilized to obtain a substantially dry radiation sterilized radioprotected mixture.

In certain preferred embodiments, one or more biologically active molecules may include one or more of protein(s), DNA and/or RNA that may be contacted with at least one soluble radioprotectant compound, for example, cysteine, glutathione, melatonin, and/or histidine, to permit the drying of the biologically active molecule(s) and the radioprotectant compound(s) to proceed at the same time, thereby to obtain a substantially dry radioprotected mixture, which may then be radiation sterilized to obtain a substantially dry radiation sterilized radioprotected mixture.

After radiation sterilization, the substantially dry radiation sterilized radioprotected mixture may be rehydrated (e.g., by resuspension and/or dissolution in water or an aqueous solvent such as a water-based buffer as would be familiar to those skilled in the biochemical, biological and/or immunological arts) to obtain a rehydrated radiation sterilized radioprotected mixture. The biological activity of the biologically active protein in the radioprotected mixture after radiation sterilization is greater (e.g., increased in a statistically significant manner relative to an appropriate control) than biological activity of a control sample of the biologically active protein that is radiation sterilized without the radioprotectant compound present. The embodiments disclosed herein thereby unexpectedly substantially protect biological activity of the biologically active protein against radiation damage during radiation sterilization.

Preferred radioprotectant compounds according to the present disclosure include cysteine, melatonin, glutathione, and histidine. In certain embodiments the radioprotectant compound may comprise one, two, three, or all four of the radioprotectant compounds cysteine, melatonin, glutathione, and histidine; in certain other embodiments the radioprotectant compound may consist of one, two, three, or all four of the radioprotectant compounds cysteine, melatonin, glutathione, and histidine. In use, the sourcing, handling, storage and solubilization of these compounds are well known and can be readily adapted to the present methods according to known methodologies and the present disclosure, including the Examples below. A radioprotectant compound as provided herein may be present in the herein described radioprotected mixture at a concentration that is effective for substantially protecting the biological activity (e.g., mitogenic activity) of a biologically active protein (e.g., mitogen such as anti-CD3 antibody, PHA, ConA, PWN) or other biologically active molecule as provided herein. Typically the radioprotectant compound may be present at a concentration of at least 0.1, 0.5, 1.0, 2.0, 3.0, 4.0, 5.0, 6.0, 7.0, 8.0, 9.0, 10, 15, 20, 25, 30, 35, 40, 45, 50, 55, 60, 65, 70, 75, 80, 85, 90, 95, 100, 200, 300, or 400 mM, including any intermediate concentration therebetween. Structures of the herein disclosed radioprotectant compounds are shown below.

Without wishing to be bound by theory, certain of the herein described radioprotectant compounds may exhibit functional properties characteristic of antioxidants and/or of free radical scavengers. The present embodiments are not, however, intended to be so limited with respect to the ability of these radioprotectant compounds to protect the herein described biologically active proteins or other biologically active molecules, and in particular the herein described biologically active proteins that are mitogens for mammalian peripheral blood lymphocytes, from compromised biological activity that would otherwise arise as the result of radiation sterilization.

L-cysteine (2-amino-3-sulfhydrylpropanoic acid) has the following structure (I):

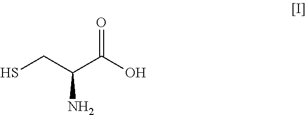

Glutathione (reduced form) (γ-L-Glutamyl-L-cysteinylglycine) has the following structure (II):

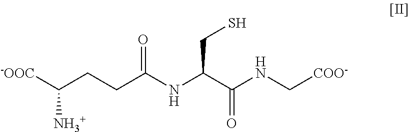

Melatonin (N-acetyl-5-methoxy tryptamine) has the following structure (III):

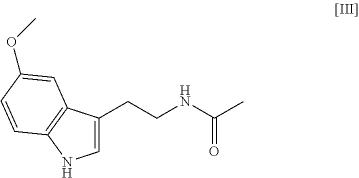 [SMILES: COc1ccc2[nH]cc(CCNC(C)=O)c2c1]

Histidine has the following structure (IV):

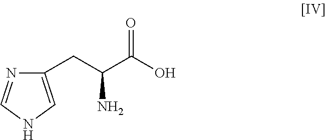 [SMILES: N[C@@H](Cc1c[nH]cn1)C(=O)O]

It will be appreciated that the practice of the several embodiments of the present invention will employ, unless indicated specifically to the contrary, conventional methods in virology, immunology, microbiology, molecular biology and recombinant DNA techniques that are within the skill of the art, and many of which are described below for the purpose of illustration. Such techniques are explained fully in the literature. See, e.g., Current Protocols in Molecular Biology or Current Protocols in Immunology, John Wiley & Sons, New York, N.Y. (2009); Ausubel et al., Short Protocols in Molecular Biology, 3^{rd} ed., Wiley & Sons, N.Y. 1995; Green and Sambrook, Molecular Cloning: A Laboratory Manual (4^{th} Ed., 2012) Cold Spring Harbor Laboratory Press, Cold Spring Harbor, N.Y.; DNA Cloning: A Practical Approach, vol. I & II (D. Glover, ed.), 2^{nd} Ed., 1995, Oxford Univ. Press, UK; Oligonucleotide Synthesis (N. Gait, ed., 1984) IRL/Oxford Univ. Press, UK; Nucleic Acid Hybridization (B. Hames & S. Higgins, eds., 1985) IRL/Oxford Univ. Press, UK; Transcription and Translation (B. Hames & S. Higgins, eds., 1984) IRL/Oxford Univ. Press, UK; Culture of Animal Cells (R. Freshney, 2010) John Wiley & Sons, N.Y.; Perbal, A Practical Guide to Molecular Cloning (1984), John Wiley & Sons, N.Y.; and other like references.

Standard techniques may be used for immunological assays including immunochemical and cellular immunological assays, and for biological sample collection and processing (e.g., blood, lymph, saliva, sputum, pus, biopsy, etc.), tissue culture and transformation (e.g., electroporation, lipofection). Immunochemical and enzymatic reactions and purification techniques may be performed using commercially available reagents according to the manufacturers' specifications or as commonly accomplished in the art, or as described herein. These and related techniques and procedures may be generally performed according to conventional methods well known in the art and as described in various general and more specific references that are cited and discussed throughout the present specification. Unless specific definitions are provided, the nomenclature utilized in connection with, and the laboratory procedures and techniques of, molecular biology, cellular and molecular immunology, biochemistry, analytical chemistry, synthetic organic chemistry, and medicinal and pharmaceutical chemistry described herein are those well known and commonly used in the art. Standard techniques may be used for recombinant technology, molecular biological and/or cellular or microbiological methodologies, chemical syntheses, chemical analyses, pharmaceutical preparation, formulation, delivery, and diagnosis and/or treatment of patients.

As used in this specification and in the appended claims, the singular forms “a,” “an” and “the” include plural references unless the content clearly dictates otherwise. Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers. Each embodiment in this specification is to be applied mutatis mutandis to every other embodiment unless expressly stated otherwise.

### Equivalents

While particular steps, elements, embodiments and applications of the present invention have been shown and described herein for purposes of illustration, it will be understood, of course, that the invention is not limited thereto since modifications may be made by persons skilled in the art, particularly in light of the foregoing teachings, without deviating from the spirit and scope of the invention. Accordingly, the invention is not limited except as by the appended claims.

### OTHER LITERATURE CITED

Meltz M L, Reiter R J, Herman T S, Kumar K S (March 1999). “Melatonin and protection from whole-body irradiation: survival studies in mice”. Mutat. Res. 425 (1): 21-7. Reiter R J, Herman T S, Meltz M L (December 1996). “Melatonin and radioprotection from genetic damage: in vivo/in vitro studies with human volunteers”. Mutat. Res. 371 (3-4): 221-8. Reiter R J, Herman T S, Meltz M L (February 1998). “Melatonin reduces gamma radiation-induced primary DNA damage in human blood lymphocytes”. Mutat. Res. 397 (2): 203-8. Tan D X, Manchester L C, Terron M P, Flores L J, Reiter R J (January 2007). “One molecule, many derivatives: a never-ending interaction of melatonin with reactive oxygen and nitrogen species?”. J. Pineal Res. 42 (1): 28-42.

Tan D X, Chen L D, Poeggeler B, Manchester L C, Reiter R J (1993). “Melatonin: a potent, endogenous hydroxyl radical scavenger”. Endocrine J. 1: 57-60. Pohanka M (2011). “Alzheimer's disease and related neurodegenerative disorders: implication and counteracting of melatonin”. Journal of Applied Biomedicine 9 (4): 185-196. Reiter R J, Manchester L C, Tan D X (September 2010). “Neurotoxins: free radical mechanisms and melatonin protection”. Curr Neuropharmacol 8 (3): 194-210. Poeggeler B, Saarela S, Reiter R J, Tan D X, Chen L D, Manchester L C, Barlow-Walden L R (November 1994). “Melatonin—a highly potent endogenous radical scavenger and electron donor: new aspects of the oxidation chemistry of this indole accessed in vitro”. Ann. N. Y. Acad. Sci. 738: 419-20. Arnao M B, Hernández-Ruiz J (May 2006). “The physiological function of melatonin in plants”. Plant Signal Behav 1 (3): 89-95. Pieri C, Marra M, Moroni F, Recchioni R, Marcheselli F (1994). “Melatonin: a peroxyl radical scavenger more effective than vitamin E”. Life Sci. 55 (15): PL271-6.

Wade et al. 1998 J. Nutritional Biochem. 9(6):308-315).

The following Examples are presented by way of illustration and not limitation.

### EXAMPLES

### Example 1

### Radioprotection of PHA Mitogenic Activity Against Radiation Sterilization

Briefly, QuantiFERON®-TB Gold Mitogen Control assay tubes were obtained from the manufacturer (QIAGEN, Inc., Germantown, Md.) and were produced by spray-drying a solution of phytohemagglutinin (PHA-P) onto the internal walls of standard blood collection tubes. The blood collection tubes were subsequently sterilized by radiation using high energy electron beam treatment (E-Beam) according to standard procedures.

Mitogenic activity, of PHA in the radiation sterilized blood collection tubes toward whole blood samples, was assessed by determining IFN-γ concentration in plasma, from whole blood samples incubated in the blood collection tubes, using QuantiFERON® ELISA according to the manufacturer's instructions (QIAGEN, Inc., Germantown, Md.). Results of one representative experiment are shown in Table 1. Radiation sterilization of spray-dried PHA on blood collection tubes, by either electron beam (e.g., E-beam) treatment or by γ-irradiation, drastically diminished the mitogenic potency of the blood collection tubes when they were tested for their ability to induce IFN-γ release by whole blood samples. The standard E-Beam treatment decreased the mitogenic activity of spray-dried PHA to about 55% of the control level. The PHA mitogenic activity declined even further following two rounds of radiation sterilization (2× E-Beam). This activity loss was proportional to the increase of radiation dosage (Table 1).

TABLE 1
QFN Mitogen Tube Potency after Treatment
Mitogenic Potency after Tx

| E-Beam | γ-Irradiation | 2× E-Beam |
|---|---|---|
| 55% | 30.8% | 29.6% |

Group mean percentages of mitogenic potency of treated QFN-Mitogen Control blood collection tubes were determined against the same lot of mitogen tubes without any treatment (100% potency).
Mitogen control tubes from a single production lot were sterilized with E-beam (16.6-30 kGy), γ-radiation (25 kGy) and 2 times of E-Beam (2× E-Beam). Mitogenic activity i.e. levels IFN-γ of the mitogen tubes were assessed with whole blood samples from a group of 11 blood donors. The mitogenic potency of sterilized tubes were presented as the group mean percentages of potency over that of the tubes without any treatment.

These results indicated that increased amounts of PHA would have to be spray-dried on each tube in order to provide radiation sterilized tubes having levels of PHA mitogenic activity that would be closer to the levels of PHA mitogenic activity in tubes that did not undergo radiation sterilization.

Candidate radioprotectant compounds were therefore selected and screened for their effects on PHA mitogenic activity. As a first selection, candidate radioprotectant compounds were identified that did not by themselves significantly alter (e.g., increase or decrease in a statistically significant manner relative to an appropriate control) mitogenic activity when included in the spray-dried PHA formulation even prior to radiation sterilization. As shown in Table 2, PHA-P formulations that contained 50 mM of the candidate radioprotectant compound cysteine, or 10 mM of the candidate radioprotectant compound melatonin, exhibited PHA mitogenic activity that was comparable to unsupplemented PHA preparations.

TABLE 2
PHA-P Activity When Formulated with Cysteine or Melatonin
PHA-P Formulation Potency with Additives

| L-Cysteine (50 mM) | Melatonin (10 mM) |
|---|---|
| 101% | 98.6% |

*Potency of PHA-P formulations with Cys and MLT were determined against the same formulation without additives (100% potency).
Potency of PHA-P formulation were assessed in whole blood samples from 6 blood donors. Results are presented as the mean percentage of the formulation potency with addtives over that without any additives.

Candidate radioprotectant compounds that did not interfere with PHA mitogenic activity were next tested for their ability to protect PHA against loss of mitogenic activity during radiation sterilization. Adding L-cysteine, reduced glutathione or melatonin into the PHA liquid formulation to 5.0 mM final concentration partially prevented the loss of PHA mitogenic activity following radiation sterilization treatments at 8.3, 16.7 and 25 kGy E-Beam (Table 3). The PHA liquid formulation in the absence of any of the candidate radioprotectant compounds (Table 3, “Control”) had only 11% of its mitogenic activity after radiation sterilization treatment at 8.3 kGy. In comparison, spray-dried PHA formulations that included L-cysteine, reduced glutathione, or melatonin exhibited 68%, 53% and 53% of the control level of mitogenic activity, respectively, following the same dosage of radiation sterilization treatment at 8.3 kGy and thus retained the substantially protected mitogenic activity.

TABLE 3
Protection of Activity Loss in PHA-P Formulation Treated with E-Beam
Group Mean Percentage of Mitogenic Activity

| E-Beam Tx | | | Glutathione | |
|---|---|---|---|---|
| (kGy) | Control | L-Cysteine | Reduced | Melatonin |
| 0.0 | 100% | 99% | 116% | 111% |
| 8.30 | 11% | 68% | 53% | 53% |
| 16.7 | 2.7% | 43% | 23% | 26% |
| 25.0 | 1.2% | 26% | 10% | 7.7% |

Group mean percentages of mitogenic activity of PHA-P formulations were determined against the control without E-Beam treatment (0.0 kGy).
Group mean activity of PHA-P formulations with additives at 5.0 mM final concentration were determined in whole blood samples from 6 blood donors. Results are presented as the mean percentages of potency against the un-treated control sample without additives.

Liquid PHA formulations containing herein-identified protective compounds also exhibited the abilities to substantially protect PHA mitogenic activity following radiation sterilization at higher dosages i.e., 16.7 and 25 kGy of E-Beam treatment.

The dose-effect protective capabilities of L-cysteine and melatonin were further assessed. L-cysteine was dissolved in a PHA-P formulation to 1.0-50 mM. Due to its lower water solubility, melatonin stock solution at 200 mM was first prepared in 100% ethyl alcohol. The stocks were then added into the PHA-P formulation to make final concentrations from 0.2-10 mM. After the E-Beam treatment, potencies of PHA-P formulations were tested with whole blood samples and mitogenic activity was determined using QuantiFERON® ELISA (QIAGEN, Inc., Germantown, Md.) according to the manufacturer's instructions.

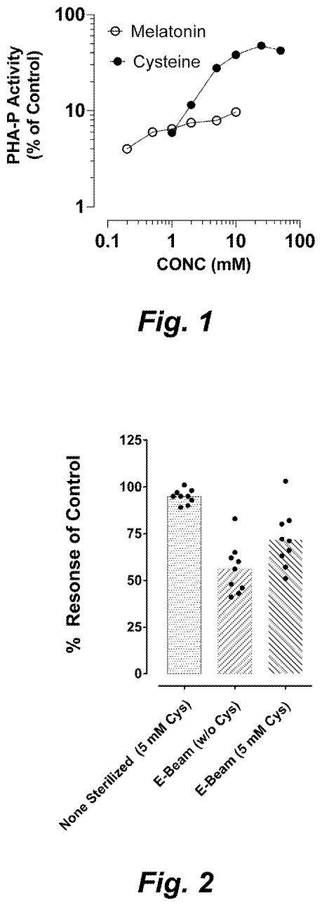

The results are summarized in FIG. 1. Increasing the concentration of L-cysteine from 1.0 mM to 10 mM in the PHA-P formulation significantly enhanced its radiation protection of PHA mitogenic activity at 25 kGy from 5.9% to 38% of the mitogenic activity level of the PHA control formulation (which contained no radioprotectant compound as an additive and was not subjected to radiation sterilization treatment).

L-cysteine was further tested for its ability to decrease the loss of PHA mitogenic potency that results from radiation sterilization of spray-dried PHA in blood collection tubes. Blood collection tubes containing spray-dried PHA (no radioprotectant compound in the spray-drying step) underwent radiation sterilization, and PHA mitogenic activity toward a whole blood sample was tested as described above. When cysteine (5 mM) was present in the spray-dried PHA formulation but the tubes did not undergo radiation sterilization, PHA mitogenic responses toward a whole blood sample averaged 94.8% of control (no radioprotectant, no radiation sterilization) levels (FIG. 2, left column) showing that cysteine did not alter the mitogen responses in non-sterilized Mitogen tubes. The radiation sterilization step decreased PHA mitogenic activity to 56% of control (no radioprotectant, no radiation sterilization) levels (FIG. 2, middle column). When cysteine (5 mM) was present in the spray-dried PHA formulation and the tubes were subjected to radiation sterilization, PHA mitogenic responses toward a whole blood sample increased from 56% (without cysteine) to 72% (5 mM cys) over the control (no radioprotectant, no radiation sterilization) level (FIG. 2, right column).

### Example 2

### Storage Stability of Radioprotectant Protection of PHA Mitogenic Activity

Mitogen (spray-dried PHA) tubes were produced and radiation sterilized as described in Example 1, comparing PHA preparations without added cysteine to PHA preparations containing 5.0 mM cysteine. The long-term storage stability of the radioprotective effect of cysteine on the mitogenic activity of spray-dried PHA was also assessed.

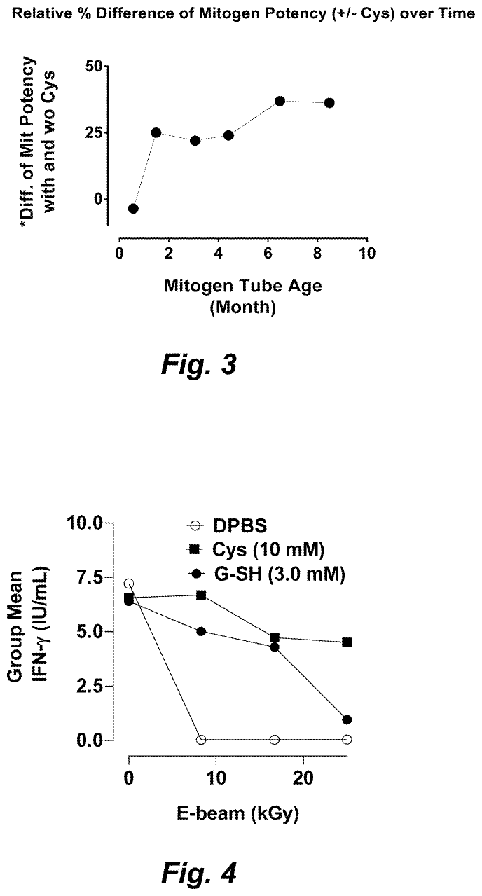

Following spray-drying and radiation sterilization the tubes were held for various time periods before being tested for mitogenic activity as described previously. At the first testing time point (within one month post production), the mitogen-containing tubes made without cysteine had similar levels of mitogenic activity to those produced with cysteine present (FIG. 3). Testing at subsequent time points extending beyond eight months post-production, however, showed that the tubes containing cysteine retained relatively higher levels of mitogenic activity than did tubes produced without cysteine.

### Example 3

### Radioprotection of Anti-CD3 Antibody T-Cell Stimulatory Activity Against Radiation Sterilization

This example describes use of a radioprotectant compound as described here to protect the activity of a biologically active antibody against the effects of radiation sterilization. In this example, materials and methods were essentially as described above in Examples 1 and 2 except as otherwise specified herein.

Anti-CD3 antibody samples were dissolved in Dulbecco's phosphate-buffered saline (DPBS) and diluted to a concentration of 66.7 μg/mL, then exposed to various doses of E-beam irradiation in the presence or absence of 10 mM cysteine (Cys) or 3 mM glutathione (G-SH). The treated antibody samples were tested for their T-cell stimulatory activity by determining their ability to induce interferon-gamma (IFNγ) secretion by T-cells present in a whole human blood sample obtained from a group of six randomly selected donors, using 0.10 μg of anti-CD3 antibody per mL of whole blood in QuantiFERON® (QFN) Nil tubes (QIAGEN, Inc., Germantown, Md.). Following the incubation of the whole blood samples with the anti-CD3 antibody in the QFN Nil tubes, blood processing, plasma harvesting and IFN-γ detection by enzyme-linked immunosorbent assay (ELISA) were performed according to the manufacturer's instructions as found in the QuantiFERON®-TB Gold Plus package insert (QFN-TB Gold Plus, QIAGEN, Inc., Germantown, Md.) except that the plasma samples were diluted 1 to 10 in ELISA kit Green Diluent immediately before testing on 8-point standard curves.

Functional anti-CD3 antibody is capable of eliciting T-cell responses to induce interferon gamma (IFN-γ) secretion in whole blood cultures. Representative results of the IFNγ responses elicited in whole blood samples by anti-CD3 antibody that was protected during E-beam irradiation with 10 mM cysteine or 3.0 mM G-SH, as compared to the unprotected control samples (irradiated in unsupplemented DPBS), are presented in FIG. 4. E-beam treatment at doses of 8.3 kGy and higher completely abolished the activity of the anti-CD3 antibody (group mean IFN-γ response in the y-axis, FIG. 4) when the antibody was irradiated in DPBS alone (open circles). In contrast, the E-beam treated anti-CD3 antibody samples that were irradiated in DPBS that also contained either 10 mM cysteine (Cys, closed squares) or 3.0 mM G-SH (closed circles), maintained stimulatory activities even at E-beam doses up to 25 kGy (FIG. 4). Therefore, both Cys and G-SH clearly protected the activities of anti-CD3 antibody treated with E-beam radiation.

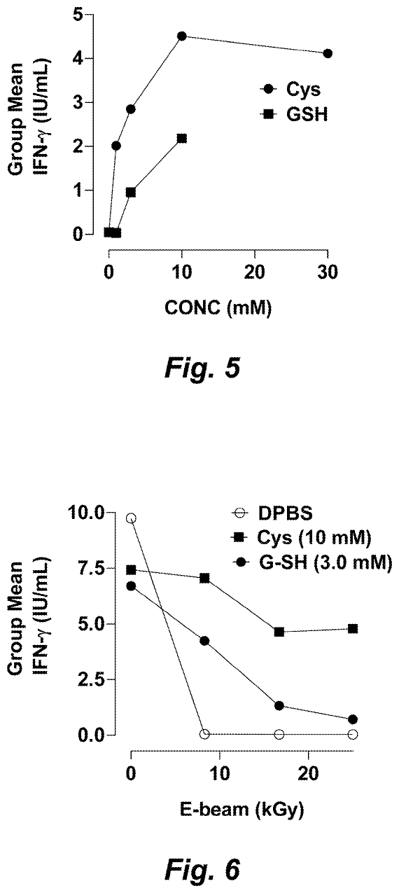

The protective effect on the anti-CD3 activity conferred by both radioprotectants, Cys and G-SH, in antibody samples that were treated with 25 kGy E-beam irradiation, exhibited a dose-dependent increase from 1.0 to 10 mM for both Cys (closed circles) and G-SH (closed squares) (FIG. 5).

### Example 4

### Radioprotection of R848 (Resiquimod) TLR Agonist Activity Against Radiation Sterilization

This example describes use of a radioprotectant compound as described here to protect the TLR agonist activity of an imidazoquinoline immune response modifier (R848) against the effects of radiation sterilization. In this example, materials and methods were essentially as described above in Examples 1-3 except as otherwise specified herein.

R848 (resiquimod, CAS 144875-48-9) is a toll-like receptor (TLR) agonist which can stimulate biological responses by natural killer (NK) cells, and its activity can be measured in the QuantiFERON® Nil tubes (QIAGEN, Inc., Germantown, Md.) QFN whole blood culture system. R848 samples were dissolved in DPBS at a concentration of 66.7 μg/mL and treated with various doses of E-beam irradiation in the presence or absence of 10 mM Cys or 3 mM G-SH. The treated R848 samples were tested for their biological activity (ability to elicit IFNγ release during an in vitro incubation) on white blood cells present in human whole blood samples, collected from a group of six randomly selected donors, using 1.0 μg R848 per mL of whole blood in QFN Nil tubes. Following incubation with the R848 samples in the QFN tubes, whole blood sample processing, plasma harvesting and IFN-γ ELISA were performed according to the QuantiFERON®-TB Gold Plus package insert (QFN-TB Gold Plus, QIAGEN, Inc., Germantown, Md.) except that the plasma samples were diluted 1:10 in ELISA kit Green Diluent immediately before testing on 8-point standard curves.

A representative result showing the radioprotective effects that were conferred on R848 samples that were irradiated in the presence of 10 mM of Cys or 3.0 mM of G-SH, as compared to R848 that was irradiated in the vehicle control (DPBS), is presented in FIG. 6. E-beam treatment at doses of 8.3 kGy and higher completely abolished the activity (group mean IFN-γ response in the y-axis FIG. 6) of R848 that was prepared in DPBS alone (open circles). In contrast, the E-beam treated R848 samples that contained either 10 mM Cys (closed squares) or 3.0 mM G-SH (closed circles), retained the stimulatory activities of R848.

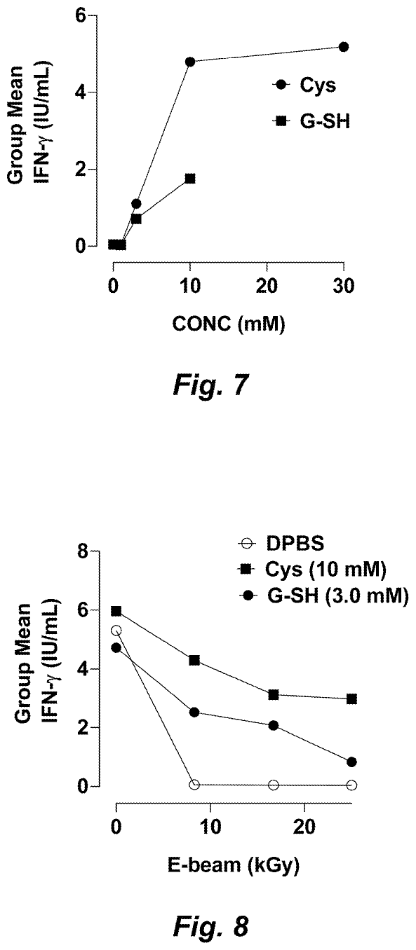

The protective effect of Cys and G-SH on the R848 activity, in samples treated with 25 kGy E-beam, exhibited a dose-dependent increase from 1.0 to 10 mM for both Cys (closed circles) and G-SH (closed squares) (FIG. 7).

### Example 5

### Radioprotection of Combined Anti-CD3 Antibody T-Cell Stimulatory Activity and R848 (Resiquimod) TLR Agonist Activity Against Radiation Sterilization

This example describes use of a radioprotectant compound as described here to protect the combined activities of a biologically active antibody and an imidazoquinoline TLR agonist immune response modifier (R848), against the effects of radiation sterilization. In this example, materials and methods were essentially as described above in Examples 1-4 except as otherwise specified herein.

QuantiFERON® Monitor (QFM) reagent (QIAGEN, Inc., Germantown, Md.), a combination of equal amounts of anti-CD3 and R848, was dissolved in DPBS at a concentration of 33.5 μg/mL and treated with various doses of E-beam irradiation in the presence or absence of 10 mM cysteine (Cys) or 3 mM glutathione (G-SH). The treated samples were tested for their biological activity (ability to elicit IFNγ release during an in vitro incubation) on white blood cells present in human whole blood samples, collected from a group of six randomly selected donors, using 0.05 μg of QFM per mL of whole blood in QFN Nil tubes. Following incubation with the QFM samples in the QFN tubes, whole blood sample processing, plasma harvesting and IFN-γ ELISA were performed according to the QuantiFERON®-TB Gold Plus package insert (QFN-TB Gold Plus, QIAGEN, Inc., Germantown, Md.) except that the plasma samples were diluted 1:10 in ELISA kit Green Diluent immediately before testing on 8-point standard curves.

Representative results showing the radioprotective effects that were conferred on QFM samples that were irradiated in the presence of 10 mM Cys or 3.0 mM of G-SH as compared to control (DPBS) are presented in FIG. 8. E-beam treatment at doses of 8.3 kGy and higher completely abolished the activity (group mean IFN-γ response in the y-axis FIG. 8) of the stimulatory anti-CD3/R848 combination that was prepared in unsupplemented DPBS alone (open circles). In contrast, the stimulatory activities of anti-CD3 and R848 were preserved in the radioprotectant-containing samples that were E-beam irradiated in PBSD containing either 10 mM Cys (closed squares) or 3.0 mM G-SH (closed circles).

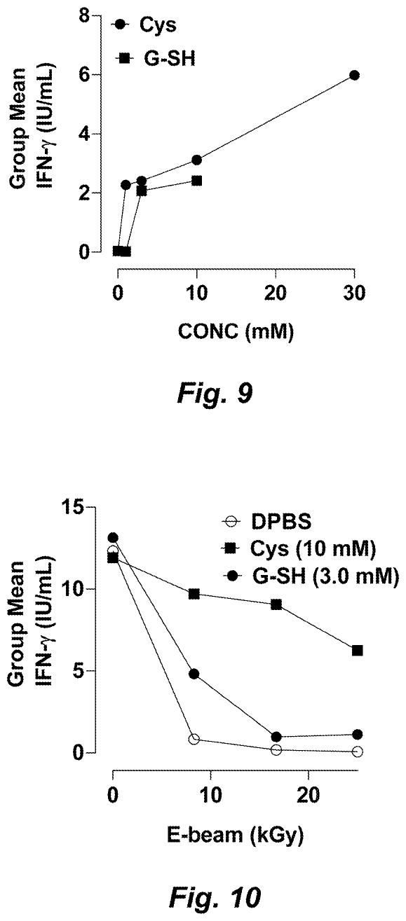

The protective effect of Cys and G-SH on the combined anti-CD3 and R848 activity, in samples treated with 25 kGy E-beam, exhibited a dose-dependent increase from 1.0 to 10 mM for both Cys (closed circles) and G-SH (closed squares) (FIG. 9).

### Example 6

### Radioprotection of Pokeweed Mitogen (PWM) Mitogenic Activity Against Radiation Sterilization

This example describes use of a radioprotectant compound as described here to protect the activity of a biologically active lectin against the effects of radiation sterilization. In this example, materials and methods were essentially as described above in Examples 1-5 except as otherwise specified herein.

Pokeweed Mitogen (PWM) samples were dissolved in DPBS at a concentration of 33.3 μg/mL and treated with various doses of E-beam irradiation in the presence or absence of 10 mM cysteine (Cys) or 3 mM glutathione (G-SH). The treated PWM samples were tested for their biological activity (ability to elicit IFNγ release during an in vitro incubation) on white blood cells present in human whole blood samples, collected from a group of six randomly selected donors, using 1.0 μg of PWM per mL of whole blood in QFN Nil tubes. Following incubation with the PWM samples in the QFN tubes, whole blood sample processing, plasma harvesting and IFN-γ ELISA were performed according to the QuantiFERON®-TB Gold Plus package insert (QFN-TB Gold Plus, QIAGEN, Inc., Germantown, Md.) except that the plasma samples were diluted 1:10 in ELISA kit Green Diluent immediately before testing on 8-point standard curves.

Representative results showing the radioprotective effects that were conferred on PWM samples that were irradiated in the presence of 10 mM Cys or 3.0 mM of G-SH as compared to control (DPBS) are presented in FIG. 10. E-beam treatment at doses of 8.3 kGy and higher completely abolished the activity (group mean IFN-γ response in the y-axis FIG. 10) of the control PWM sample that was prepared and irradiated in unsupplemented DPBS alone (open circles). In contrast, the stimulatory activity of PWM was preserved in the radioprotectant-containing samples that were E-beam irradiated in PBSD containing either 10 mM Cys (closed squares) or 3.0 mM G-SH (closed circles).

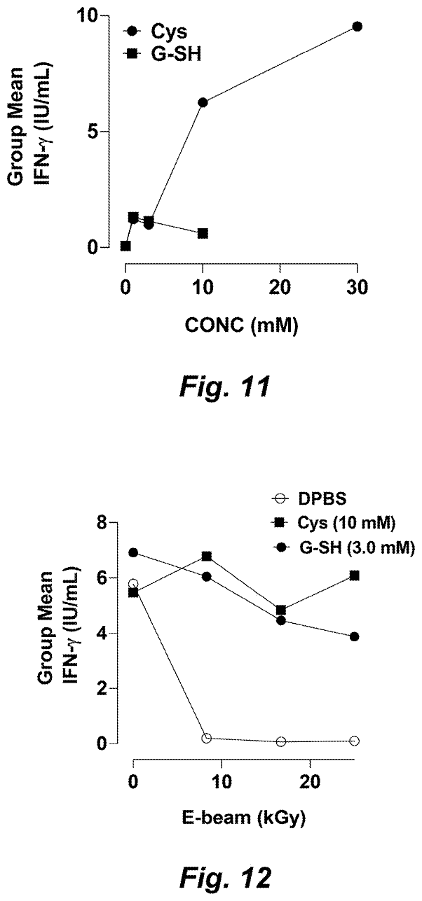

The protective effect of Cys and G-SH on PWM activity, in samples treated with 25 kGy E-beam, exhibited a dose-dependent increase from 1.0 to 10 mM for both Cys (closed circles) and G-SH (closed squares) (FIG. 11).

### Example 7

### Radioprotection of Concanavalin A (ConA) Mitogenic Activity Against Radiation Sterilization

This example describes use of a radioprotectant compound as described here to protect the activity of a biologically active lectin against the effects of radiation sterilization. In this example, materials and methods were essentially as described above in Examples 1-6 except as otherwise specified herein.

Concanavalin A (ConA) is known as a lectin which activates T-lymphocytes. ConA samples were dissolved in DPBS at a concentration of 3333 μg/mL and treated with various doses of E-beam irradiation in the presence or absence of 10 mM cysteine (Cys) or 3 mM glutathione (G-SH).

The treated ConA samples were tested for their biological activity (ability to elicit IFNγ release during an in vitro incubation) on white blood cells present in human whole blood samples, collected from a group of six randomly selected donors, using 100 μg of ConA per mL of whole blood in QFN Nil tubes. Following incubation with the ConA samples in the QFN tubes, whole blood sample processing, plasma harvesting and IFN-γ ELISA were performed according to the QuantiFERON®-TB Gold Plus package insert (QFN-TB Gold Plus, QIAGEN, Inc., Germantown, Md.) except that the plasma samples were diluted 1:10 in ELISA kit Green Diluent immediately before testing on 8-point standard curves.

Representative results showing the radioprotective effects that were conferred on ConA samples that were irradiated in the presence of 10 mM Cys or 3.0 mM of G-SH as compared to control (DPBS) are presented in FIG. 12. E-beam treatment at doses of 8.3 kGy and higher completely abolished the activity (group mean IFN-γ response in the y-axis FIG. 12) of the control ConA sample that was prepared and irradiated in unsupplemented DPBS alone (open circles). In contrast, the stimulatory activity of ConA was preserved in the radioprotectant-containing samples that were E-beam irradiated in PBSD containing either 10 mM Cys (closed squares) or 3.0 mM G-SH (closed circles).

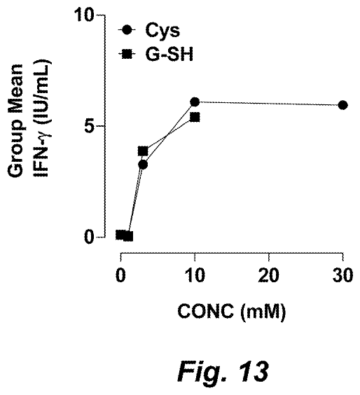

The protective effect of Cys and G-SH on ConA activity, in samples treated with 25 kGy E-beam, exhibited a dose-dependent increase from 1.0 to 10 mM for both Cys (closed circles) and G-SH (closed squares) (FIG. 13).

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

1. A method of protecting biological activity of a biologically active molecule against radiation damage during radiation sterilization, comprising:
(a) contacting the biologically active molecule in an aqueous solution with at least one soluble radioprotectant compound to obtain a radioprotected mixture prior to radiation sterilization; and
(b) radiation sterilizing the radioprotected mixture, wherein biological activity of the biologically active molecule in the radioprotected mixture after radiation sterilization is greater than biological activity of a control sample of the biologically active molecule that is radiation sterilized without the radioprotectant compound present, and thereby protecting biological activity of the biologically active molecule against radiation damage during radiation sterilization.

2. The method of claim 1 wherein the radioprotected mixture is dried prior to the step of radiation sterilizing.

3. A method of protecting a plurality of molecules of a biologically active molecule against a loss of biological activity from said plurality of molecules during a period of time in storage, comprising:
(a) contacting the biologically active molecule in an aqueous solution with at least one soluble radioprotectant compound to obtain a radioprotected mixture prior to radiation sterilization;
(b) drying the radioprotected mixture to obtain a dried radioprotected mixture;
(c) radiation sterilizing the dried radioprotected mixture; and
(d) storing the dried radioprotected mixture for a period of time to obtain a stored dried radioprotected mixture, wherein biological activity of the biologically active molecule in the stored dried radioprotected mixture after radiation sterilization and storage for said period of time is greater than biological activity of a control sample of the biologically active molecule that is dried, radiation sterilized without the radioprotectant compound present, and then stored for the period of time, and thereby protecting a plurality of molecules of the biologically active molecule against loss of biological activity during the period of time in storage.

4. A method of protecting biological activity of a biologically active molecule against radiation damage during radiation sterilization, comprising:
(a) contacting the biologically active molecule in an aqueous solution with at least one soluble radioprotectant compound to obtain a radioprotected mixture prior to radiation sterilization;
(b) drying the radioprotected mixture to obtain a dried radioprotected mixture; and
(c) radiation sterilizing the dried radioprotected mixture to obtain a dried radiation sterilized radioprotected mixture, wherein, following rehydration of the dried radiation sterilized radioprotected mixture to obtain a rehydrated radiation sterilized radioprotected mixture, biological activity of the biologically active molecule in the radioprotected mixture after radiation sterilization is greater than biological activity of a control sample of the biologically active molecule that is radiation sterilized without the radioprotectant compound present, and thereby protecting biological activity of the biologically active molecule against radiation damage during radiation sterilization.

5. The method of claim 1, wherein the biologically active molecule comprises one or more of (i) a biologically active protein, (ii) a mitogen, (iii) an antibody, (iv) an enzyme, (v) a cytokine, (vi) a growth factor, (vii) a hormone and (viii) a biologically active imidazoquinoline having TLR agonist activity.

6. The method of claim 5 wherein the mitogen is selected from phytohemagglutinin (PHA), concanavalin A (ConA), and pokeweed mitogen (PWM).

7. The method of claim 1, wherein the radioprotectant compound comprises at least one antioxidant compound.

8. The method of claim 7 wherein the antioxidant compound is selected from cysteine, glutathione and melatonin.

9. The method of claim 1, wherein the radioprotectant compound comprises histidine.

10. The method of claim 1, wherein the radioprotectant compound is present in the radioprotected mixture at a concentration of at least 0.1, 0.2, 0.3, 0.5, 0.7, 1.0, 2, 3, 4, 5, 6, 7, 8, 9, 10, 11, 12, 13, 14, 15, 16, 17, 18, 19, 20, or 50 millimolar.

11. The method of claim 1, wherein the biological activity comprises mitogenic activity.

12. The method of claim 11 wherein the mitogenic activity comprises lymphocyte proliferation inducing activity.

13. The method of claim 12 wherein the lymphocyte proliferation activity comprises T-cell proliferation inducing activity.

14. The method of claim 5 wherein the biologically active imidazoquinoline having TLR agonist activity comprises one or more of imiquimod, gardiquimod, and resiquimod (R848).

15. A method of protecting biological activity of a biologically active protein against radiation damage during radiation sterilization, comprising:
(a) contacting the biologically active protein in an aqueous solution with at least one soluble radioprotectant compound to obtain a radioprotected mixture prior to radiation sterilization; and
(b) radiation sterilizing the radioprotected mixture, wherein biological activity of the biologically active protein in the radioprotected mixture after radiation sterilization is greater than biological activity of a control sample of the biologically active protein that is radiation sterilized without the radioprotectant compound present, and thereby protecting biological activity of the biologically active protein against radiation damage during radiation sterilization.

16. The method of claim 15 wherein the radioprotected mixture is dried prior to the step of radiation sterilizing.

17. A method of protecting a plurality of molecules of a biologically active protein against a loss of biological activity from said plurality of molecules during a period of time in storage, comprising:
(a) contacting the biologically active protein in an aqueous solution with at least one soluble radioprotectant compound to obtain a radioprotected mixture prior to radiation sterilization;
(b) drying the radioprotected mixture to obtain a dried radioprotected mixture;
(c) radiation sterilizing the dried radioprotected mixture; and
(d) storing the dried radioprotected mixture for a period of time to obtain a stored dried radioprotected mixture, wherein biological activity of the biologically active protein in the stored dried radioprotected mixture after radiation sterilization and storage for said period of time is greater than biological activity of a control sample of the biologically active protein that is dried, radiation sterilized without the radioprotectant compound present, and then stored for the period of time, and thereby protecting a plurality of molecules of the biologically active protein against loss of biological activity during the period of time in storage.

18. A method of protecting biological activity of a biologically active protein against radiation damage during radiation sterilization, comprising:
(a) contacting the biologically active protein in an aqueous solution with at least one soluble radioprotectant compound to obtain a radioprotected mixture prior to radiation sterilization;
(b) drying the radioprotected mixture to obtain a dried radioprotected mixture; and
(c) radiation sterilizing the dried radioprotected mixture to obtain a dried radiation sterilized radioprotected mixture, wherein, following rehydration of the dried radiation sterilized radioprotected mixture to obtain a rehydrated radiation sterilized radioprotected mixture, biological activity of the biologically active protein in the radioprotected mixture after radiation sterilization is greater than biological activity of a control sample of the biologically active protein that is radiation sterilized without the radioprotectant compound present, and thereby protecting biological activity of the biologically active protein against radiation damage during radiation sterilization.

19. The method of claim 15, wherein the biologically active protein comprises one or more of (i) a mitogen, (ii) an antibody, (iii) an enzyme, (iv) a cytokine, (v) a growth factor, and (vi) a hormone.

20. The method of claim 19 wherein the mitogen is selected from phytohemagglutinin (PHA), concanavalin A (ConA), and pokeweed mitogen (PWM).

21. The method of claim 15, wherein the radioprotectant compound comprises at least one antioxidant compound.

22. The method of claim 21 wherein the antioxidant compound is selected from cysteine, glutathione and melatonin.

23. The method of claim 15, wherein the radioprotectant compound comprises histidine.

24. The method of claim 15, wherein the radioprotectant compound is present in the radioprotected mixture at a concentration of at least 0.1, 0.2, 0.3, 0.5, 0.7, 1.0, 2, 3, 4, 5, 6, 7, 8, 9, 10, 11, 12, 13, 14, 15, 16, 17, 18, 19, 20, or 50 millimolar.

25. The method of claim 15, wherein the biological activity comprises mitogenic activity.

26. The method of claim 25 wherein the mitogenic activity comprises lymphocyte proliferation inducing activity.

27. The method of claim 26 wherein the lymphocyte proliferation activity comprises T-cell proliferation inducing activity.
